(19) United States
(12) Patent Application Publication
(10) Pub. No.: US 2019/0021677 A1
(43) Pub. Date: Jan. 24, 2019

(54) METHODS AND SYSTEMS FOR CLASSIFICATION AND ASSESSMENT USING MACHINE LEARNING

(71) Applicant: Siemens Healthcare GmbH, Erlangen (DE)

(72) Inventors: Sasa Grbic, Princeton, NJ (US); Amitkumar Bhupendrakumar Shah, Erlangen (DE); Grzegorz Soza, Heroldsberg (DE); Philipp Hoelzer, Bubenreuth (DE); Eva Eibenberger, Nuernberg (DE); Stefan Grosskopf, Nuernberg (DE); Michael Suehling, Erlangen (DE)

(73) Assignee: Siemens Healthcare GmbH, Erlangen (DE)

(21) Appl. No.: 15/821,883

(22) Filed: Nov. 24, 2017

Related U.S. Application Data
(60) Provisional application No. 62/533,681, filed on Jul. 18, 2017.

(51) Int. Cl. A61B 5/00; G06K 9/62; G06T 7/00; A61B 6/03; A61B 5/02; A61B 6/00; A61B 8/08

(52) U.S. Cl. CPC A61B 5/7267; G06K 9/6267; G06T 7/0012; A61B 6/032; A61B 6/037; A61B 5/02042; G06T 2207/30096; A61B 8/5223; G06T 2207/10081; G06T 2207/20081; G06T 2207/30012; G06T 2207/30016; A61B 6/5217

(57) ABSTRACT

In one example embodiment, a method for assessing a patient include determining scan parameters of the patient using deep learning, scanning the patient using the determining scan parameters to generate at least one three-dimensional (3D) image, detecting an injury from the 3D image using the deep learning, classifying the detected injury using the deep learning and assessing a criticality of the detected injury based on the classifying using the deep learning.

### PRIORITY

This application claims priority to U.S. Provisional Application No. 62/533,681, the entire contents of which are hereby incorporated by reference.

### BACKGROUND

Computed tomography (CT) is an imaging modality used for rapid diagnosis of traumatic injuries with high sensitivity and specificity.

In a conventional trauma workflow, plain radiographs and focused assessment with sonography for trauma (FAST) are done and then hemodynamically stable patients are scanned for selective anatomical regions with CT.

Polytrauma patients, such as those from motor vehicle accidents, falls from great heights and penetrating trauma may be subject to whole body computed tomography (WBCT).

CT angiography (CTA) is used for diagnosis of vascular injuries. Abdomen and pelvis injuries are better diagnosed with biphasic contrast scan (arterial and portal venous phases) or with a split bolus technique. Delayed phase is recommended for urinary track injuries. These scans are often done based on the injured anatomical region, for example, head, neck, thorax, abdomen and pelvis. In addition, extremities are also scanned if corresponding injuries are suspected.

Each anatomical region scan may be reconstructed with specific multiplanar reformats (MPR), gray level windows and kernels. For example, axial, sagittal and coronal MPR are used for spine with bone and soft tissue kernel. In addition, thin slice reconstructions are used for advanced post processing such as 3D rendering and image based analytics. In addition, some radiologists also use dual energy scans for increased confidence in detection of a hemorrhage, solid organ injuries, bone fractures and virtual bone removal. Thus, there could be more than 20 image reconstructions and thousands of images in one examination.

In some highly optimized emergency departments (ED) that have a dedicated CT scanner, emergency radiologists do a primary image read with first few reconstructions close to the CT acquisition workplace or in a separate reading room in order to give a quick report on life threatening injuries for treatment decisions and deciding on need for additional imaging studies. This is followed by a more exhaustive secondary reading to report on all other findings.

In some hospitals where radiologists do an image read for multiple remote scanners, the imaging study may be divided into sub-specialties. For example, head & neck images are read by a neuroradiologist, chest/abdomen/pelvis by body radiologists and extremities by musculoskeletal (MSK) radiologists.

In certain circumstances, repeated follow-up CT scans are done after several hours for monitoring injuries.

### SUMMARY

Diagnosing traumatic/polytraumatic injuries brings about special challenges: (1) diagnosis has to be accurate and fast for interventions to be efficacious, (2) a high CT image data volume has to be processed and (3) conditions can be life-threatening and hence critically rely on proper diagnosis and therapy.

During the reading of the CT image data volume, the radiologist reads a high number of images within a short time. Due to a technical advancement in the image acquisition devices like CT scanners, a number of images generated has increased. Thus, reading the high number of images has become a tedious task. Within the images, the radiologist finds and assesses the location and extent of injuries, in addition to inspecting present anatomical structures in the images.

Some of the conditions or injuries can be life-threatening. Thus, a time to read and diagnose images of trauma patients should be reduced. Reducing the overall time for diagnosis would help to increase the probability of patient survival. The data overload sometimes also leads to unintentional missing of injuries that might also have critical consequences on patient management.

Moreover, special types of injuries are wounds created by bullets, knives or other objects penetrating the body. Currently, there is no dedicated support for making diagnosis for such wounds during the reading by the radiologist.

At least one example embodiment provides a method for assessing a patient. The method includes determining scan parameters of the patient using machine learning, scanning the patient using the determined scan parameters to generate at least one three-dimensional (3D) image, detecting an injury from the 3D image using the machine learning, classifying the detected injury using the machine learning and assessing a criticality of the detected injury based on the classifying using the machine learning.

In at least one example embodiment, the method further includes quantifying the classified injury, the assessing assesses the criticality based on the quantifying.

In at least one example embodiment, the quantifying includes determining a volume of the detected injury using the machine learning.

In at least one example embodiment, the quantifying includes estimating a total blood loss using the machine learning.

In at least one example embodiment, the method further includes selecting one of a plurality of therapeutic options based on the assessed criticality using the machine learning.

In at least one example embodiment, the method further includes displaying the detected injury in the image and displaying the assessed criticality over the image.

In at least one example embodiment, the displaying the assessed criticality includes providing an outline around the detected injury, a weight of the outline representing the assessed criticality.

At least another example embodiment provides a system including a memory storing computer-readable instructions and a processor configured to execute the computer-readable instructions to determine scan parameters of a patient using machine learning, obtain a three-dimensional (3D) image of the patient, the 3D image being generated from the determined scan parameters, detect an injury from the 3D image using the machine learning, classify the detected injury using the machine learning, and assess a criticality of the detected injury based on the classifying using the machine learning.

In at least one example embodiment, the processor is configured to execute the computer-readable instructions to quantify the classified injury, the assessed criticality being based on the quantification.

In at least one example embodiment, the processor is configured to execute the computer-readable instructions to determine a volume of the detected injury using the machine learning.

In at least one example embodiment, the processor is configured to execute the computer-readable instructions to estimate a total blood loss using the machine learning.

In at least one example embodiment, the processor is configured to execute the computer-readable instructions to select one of a plurality of therapeutic options based on the assessed criticality using the machine learning.

In at least one example embodiment, the processor is configured to execute the computer-readable instructions to display the detected injury in the image and display the assessed criticality over the image.

In at least one example embodiment, the processor is configured to execute the computer-readable instructions to display the assessed criticality by providing an outline around the detected injury, a weight of the outline representing the assessed criticality.

### BRIEF DESCRIPTION OF THE DRAWINGS

Example embodiments will be more clearly understood from the following detailed description taken in conjunction with the accompanying drawings. FIGS. 1-15 represent non-limiting, example embodiments as described herein.

FIG. 1 illustrates a computed tomography (CT) system 1 according to at least one example embodiment;

FIG. 2 illustrates the control system 100 of FIG. 1 according to an example embodiment;

FIG. 3 illustrates a method of using an intelligent post-processing workflow which facilitates reading of medical images for trauma diagnosis according to an example embodiment;

FIG. 4 illustrates a display which correlates geometrical properties to findings according to an example embodiment;

FIG. 5 illustrates a method of utilizing the machine/deep learning network for certain body regions, according to an example embodiment;

FIG. 6 illustrates an example embodiment of assessing the criticality of an injury in the head;

FIG. 7 illustrates an example embodiment of determining a therapy;

FIG. 8 illustrates an example embodiment of detecting traumatic bone marrow lesions in the spine;

FIG. 9 illustrates an example embodiment of detecting a spinal cord in a patient;

FIG. 10 illustrates an example embodiment of classifying a spinal fracture;

FIG. 11 illustrates an example embodiment of detecting a cardiac contusion;

FIG. 12 illustrates an example embodiment of detection, classification, quantification and a criticality assessment of a hematoma on the spleen, liver or kidney;

FIG. 13 illustrates a method for training the machine/deep learning network according to an example embodiment;

FIG. 14 illustrates an example embodiment of a user interface; and

FIG. 15 illustrates an example embodiment of an interactive checklist generated by the system of FIG. 1.

### DETAILED DESCRIPTION

Various example embodiments will now be described more fully with reference to the accompanying drawings in which some example embodiments are illustrated.

Accordingly, while example embodiments are capable of various modifications and alternative forms, embodiments thereof are shown by way of example in the drawings and will herein be described in detail. It should be understood, however, that there is no intent to limit example embodiments to the particular forms disclosed, but on the contrary, example embodiments are to cover all modifications, equivalents, and alternatives falling within the scope of the claims. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of example embodiments and corresponding detailed description are presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments will be described with reference to acts and symbolic representations of operations (e.g., in the form of flowcharts) that may be implemented as program modules or functional processes including routines, programs, objects, components, data structures, etc., that perform particular tasks or implement particular abstract data types and may be implemented using existing hardware at existing elements or control nodes. Such existing hardware may include one or more Central Processing Units (CPUs), system on chips (SoCs), digital signal processors (DSPs), application-specific-integrated-circuits, field programmable gate arrays (FPGAs) computers or the like.

Unless specifically stated otherwise, or as is apparent from the discussion, terms such as “processing” or “computing” or “calculating” or “determining” or “displaying” or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

Note also that the software implemented aspects of example embodiments are typically encoded on some form of tangible (or recording) storage medium. The tangible storage medium may be read only, random access memory, system memory, cache memory, magnetic (e.g., a floppy disk, a hard drive, MRAM), optical media, flash memory, buffer, combinations thereof, or other devices for storing data or video information magnetic (e.g., a hard drive) or optical (e.g., a compact disk read only memory, or “CD ROM”). Example embodiments are not limited by these aspects of any given implementation and include cloud-based storage.

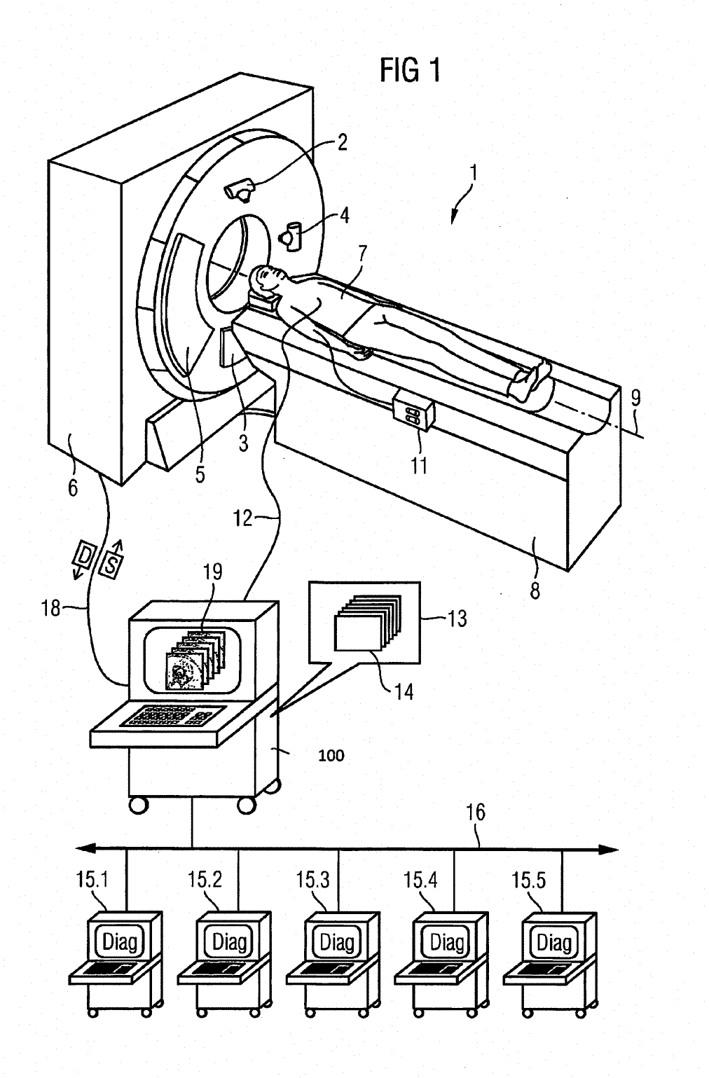

FIG. 1 illustrates a computed tomography (CT) system 1 according to at least one example embodiment. While a CT system is described, it should be understood that example embodiments may be implemented in other medical imaging devices, such as a diagnostic or therapy ultrasound, x-ray, magnetic resonance, positron emission, or other device.

The CT system 1 includes a first emitter/detector system with an x-ray tube 2 and a detector 3 located opposite it. Such a CT system 1 can optionally also have a second x-ray tube 4 with a detector 5 located opposite it. Both emitter/detector systems are present on a gantry, which is disposed in a gantry housing 6 and rotates during scanning about a system axis 9.

If two emitter/detector systems are used, it is possible to achieve increased temporal resolution for supplementary cardio examinations or it is possible to scan with different energies at the same time, so that material breakdown is also possible. As a result, supplementary examination information can be supplied in the body regions under consideration.

A traumatized patient 7 is positioned on a movable examination couch 8, which can be moved along the system axis 9 through the scan field present in the gantry housing 6, in which process the attenuation of the x-ray radiation emitted by the x-ray tubes is measured by the detectors. A whole-body topogram may be recorded first, a z-distribution to different body regions takes place and respectively reconstructed CT image data is distributed individually by way of a network 16 to specialist diagnostic workstations 15.x in each instance for the respective diagnosis of relevant for the body regions.

In an example embodiment, a whole-body CT is performed but a contrast agent bolus can also be injected into the patient 7 with the aid of a contrast agent applicator 11, so that blood vessels can be identified more easily. For cardio recordings, heart activity can also be measured using an EKG line 12 and an EKG-gated scan can be performed.

The CT system 1 is controlled by a control system 100 and the CT system 1 is connected to the control system 100 by a control and data line 18. Raw data D from the detectors 3 and 5 are sent to the control system 100 through the control and data line 18 and the control commands S are transferred from the control system 100 to the CT system 1 through the control and data line 18.

Present in a memory 103 of the control system 100 are computer programs 14, which, when executed cause the control system 100 to perform operate the CT system 1.

CT image data 19, in particular also the topogram, can additionally be output by the control system 100, it being possible to assist the distribution of the body regions by way of manual inputs.

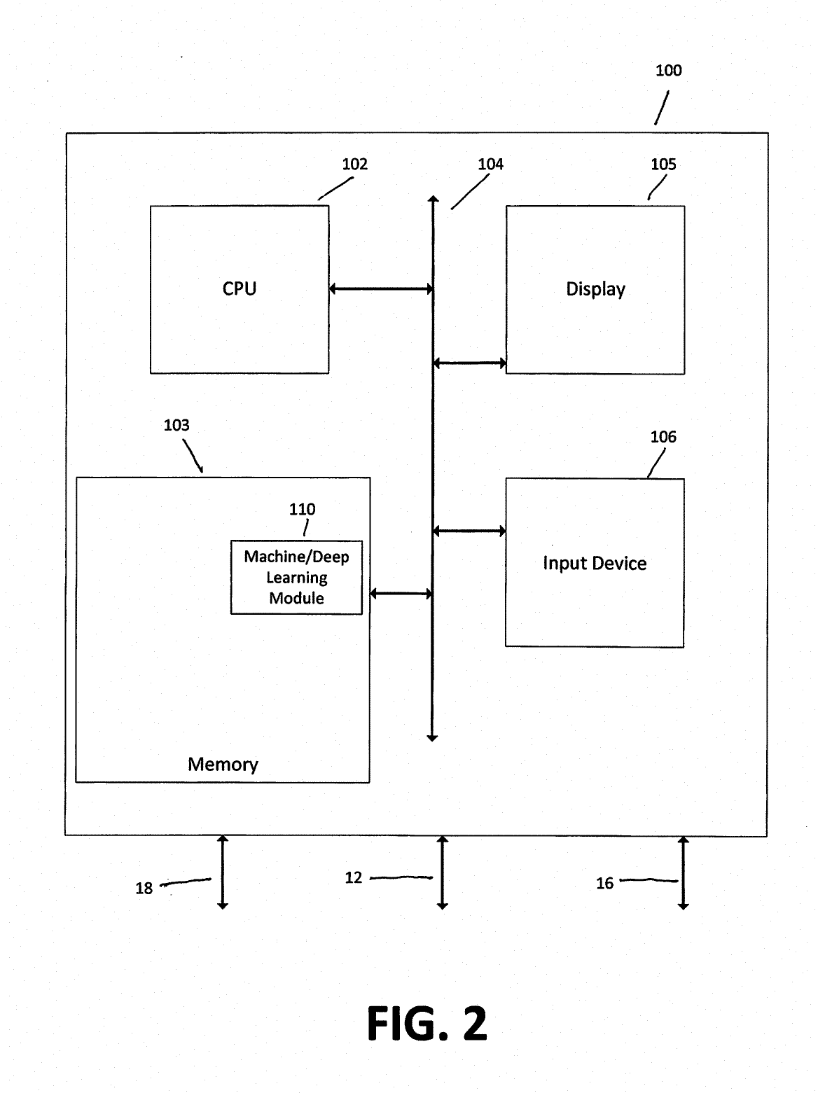

FIG. 2A illustrates the control system 100 of FIG. 1 according to an example embodiment. The control system 100 may include a processor 102, a memory 103, a display 105 and input device 106 all coupled to an input/output (I/O) interface 104.

The input device 106 may be a singular device or a plurality of devices including, but not limited to, a keyboard, trackball, mouse, joystick, touch screen, knobs, buttons, sliders, touch pad, and combinations thereof. The input device 106 generates signals in response to user action, such as user pressing of a button.

The input device 106 operates in conjunction with a user interface for context based user input. Based on a display, the user selects with the input device 106 one or more controls, rendering parameters, values, quality metrics, an imaging quality, or other information. For example, the user positions an indicator within a range of available quality levels. In alternative embodiments, the processor 102 selects or otherwise controls without user input (automatically) or with user confirmation or some input (semi-automatically).

The memory 103 is a graphics processing memory, video random access memory, random access memory, system memory, cache memory, hard drive, optical media, magnetic media, flash drive, buffer, combinations thereof, or other devices for storing data or video information. The memory 103 stores one or more datasets representing a three-dimensional volume for segmented rendering.

Any type of data may be used for volume rendering, such as medical image data (e.g., ultrasound, x-ray, computed tomography, magnetic resonance, or positron emission). The rendering is from data distributed in an evenly spaced three-dimensional grid, but may be from data in other formats (e.g., rendering from scan data free of conversion to a Cartesian coordinate format or scan data including data both in a Cartesian coordinate format and acquisition format). The data is voxel data of different volume locations in a volume. The voxels may be the same size and shape within the dataset or the size of such a voxel can be different in each direction (e.g., anisotropic voxels). For example, voxels with different sizes, shapes, or numbers along one dimension as compared to another dimension may be included in a same dataset, such as is associated with anisotropic medical imaging data. The dataset includes an indication of the spatial positions represented by each voxel.

The dataset is provided in real-time with acquisition. For example, the dataset is generated by medical imaging of a patient using the CT system 1. The memory 103 stores the data temporarily for processing. Alternatively, the dataset is stored from a previously performed scan. In other embodiments, the dataset is generated from the memory 103, such as associated with rendering a virtual object or scene. For example, the dataset is an artificial or “phantom” dataset.

The processor 102 is a central processing unit, control processor, application specific integrated circuit, general processor, field programmable gate array, analog circuit, digital circuit, graphics processing unit, graphics chip, graphics accelerator, accelerator card, combinations thereof, or other developed device for volume rendering. The processor 102 is a single device or multiple devices operating in serial, parallel, or separately. The processor 102 may be a main processor of a computer, such as a laptop or desktop computer, may be a processor for handling some tasks in a larger system, such as in an imaging system, or may be a processor designed specifically for rendering. In one embodiment, the processor 102 is, at least in part, a personal computer graphics accelerator card or components, such as manufactured by nVidia®, ATI™, Intel® or Matrox™.

The processor 102 is configured to perform a method of using an intelligent post-processing workflow which facilitates reading of medical images for trauma diagnosis as will be described in greater detail below by executing computer-readable instructions stored in the memory 103.

Different platforms may have the same or different processor 102 and associated hardware for segmented volume rendering. Different platforms include different imaging systems, an imaging system and a computer or workstation, or other combinations of different devices. The same or different platforms may implement the same or different algorithms for rendering. For example, an imaging workstation or server implements a more complex rendering algorithm than a personal computer. The algorithm may be more complex by including additional or more computationally expensive rendering parameters.

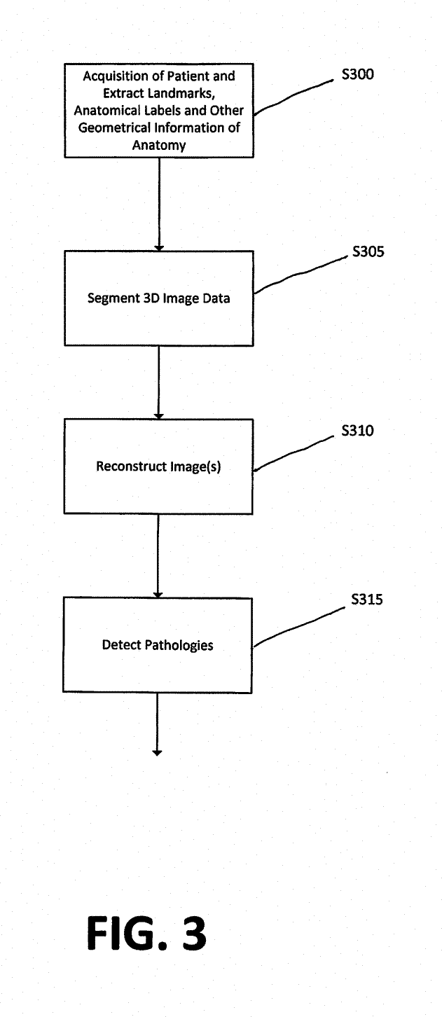

The memory 103 stores a machine/deep learning module 110, which includes computer-readable instructions for performing intelligent post-processing workflow described in herein, such as the method described with reference to FIG. 3.

The processor 102 may be hardware devices for accelerating volume rendering processes, such as using application programming interfaces for three-dimensional texture mapping. Example APIs include OpenGL and DirectX, but other APIs may be used independent of or with the processor 102. The processor 102 is operable for volume rendering based on the API or an application controlling the API. The processor may also have vector extensions (like AVX2 or AVX512) that allow an increase of the processing speed of the rendering.

FIG. 3 illustrates a method of using an intelligent post-processing workflow which facilitates reading of medical images for trauma diagnosis. The method of FIG. 3 can be performed by the CT system 1 including the control system 100.

Today's reading process is time-consuming and consists of multiple manual steps. Reading physicians read acquired data either as 2D images or they use multi-planar reconstructions (MPRs). During the reading process, they go manually from one anatomical structure (e.g., an organ) to another. For each structure, the reading physician chooses and load the best data manually (e.g., loading images with a sharp kernel to assess bones) to assess a given structure. Within the structure, the reading physician scrolls up and down and/or rotates image/reference lines several times to obtain views which to read this body part. In addition, for each examined structure, the reading physician manually adjusts manually visualization parameters like windowing, slab thickness, intensity projection, etc. This helps to obtain visualization for a given structure, thus delivering improved reading results. For better viewing, some slices can be put together to form a slab that is at least of the thickness of the original slices, but can be adjusted to be higher.

However, all of these tasks are time consuming. Also, the amount of data used costs time needed for image reconstruction or for image transfer.

In the context of trauma, reducing processing and reading time can be translated into increasing the probability of patient survival.

This reading process consisting of multiple manual steps is time consuming. To reduce this time, the inventors have discovered an intelligent post-processing workflow which facilitates reading of medical images for trauma diagnosis.

Referring back to FIG. 3, the steps illustrated in FIG. 3 do not necessarily need to be performed in the exact same order as listed below. The steps shown in FIG. 3 may be performed by the processor 102 executing computer-readable instructions stored in the memory 103.

As shown in FIG. 3, a camera and/or a scanner (e.g., the detectors 3 and 5) generates raw image data of a patient at S300 and the system acquires the raw image data. As will be described below, the acquisition of a patient may include acquiring two sets of image data: image data associated with an initial scan (a first image) (e.g., performed by a camera) and the raw 3D image data generated from an actual scan performed by the scanner (at least one second image), or just the raw 3D image data generated from the actual scan performed by the scanner (e.g., CT). The camera and the scanner are distinct objects. The camera may be an optical camera (e.g., photo camera, camcorder, depth camera such Microsfot Kinect). These cameras capture images directly without any intermediate reconstruction algorithm as in CT images and provide information about the surface of the object/patient. CT scanners use body penetrating radiation to reconstruct an image of the patient's interior. In the case of penetrating trauma the camera may show an entry point and the CT scanner shows a trajectory of the penetrating object within the body.

The image data may be slices of data of a whole body of the patient or a particular section of the body covering one or many anatomical features.

For example, the acquired 3D image data can consist of 1 or n scans each having 1 or m reconstructions (which are performed at S310). Each scan can comprise one part of the body (e.g. head or thorax) reconstructed in multiple ways (e.g., using different kernels and/or different slice thickness for the same body region) or one scan can cover a whole body of the patient.

In order to reduce the amount of data to be processed and transferred to a reading workstation 15.k and to improve the visualization for the reading, the system 100 selects a portion of the image data and processes the selected portion of the image data as will be described below.

At S300, the processor extracts landmark coordinates ((x,y) or (x,y,z)), anatomical labels (e.g., vertebra labels) and other geometrical information on the anatomy (e.g., centerlines of vessels, spine, bronchia, etc.) within the selected image data using the machine/deep learning network based on a set of previously annotated data. The data extracted at S300 may be referred to in general as anatomical information.

The landmarks to be extracted are stored as a list of landmarks in the memory 103 based on the selected image data. The anatomical labels may not have precise coordinates, but are associated with a region in the image.

For the purposes of the present application, machine learning and deep learning may be used interchangeably.

The machine/deep learning may be implemented by the processor and may be a convolutional neural network, a recurrent neural network with long short-term memory, a generative adversarial network, a Siamese network or reinforcement learning. The machine/deep learning network may be trained using labeled medical images that were read by a human as will be described in greater detail below.

Different machine/deep learning networks may be implemented by the processor based on the implementation of the method of FIG. 3. For example, the convolutional neural network may be used to detect localized injuries (e.g., fractures) due to its ability to detect patch wise features and classify patches, the recurrent neural network with long short-term memory may be used to segment structures with recurrent substructures (e.g., spine, ribcage, teeth) due to its ability to provide a spatial or temporal context between features and temporal or spatial constraints, the generative adversarial network may be used for segmentation or reconstruction due to its ability to add shape constraints, Siamese networks may be used to distinguish between a normality and abnormality and detect deviations from symmetry (e.g., brain injuries) due to its ability to establish relationships and distances between images and reinforcement learning may be used for navigation, bleeding and bullet trajectories due to its ability to provide sparse time-delayed feedback.

Based on the information from the admission of the patient, a machine/deep learning algorithm determines how to triage a patient for an appropriate modality and subsequently determines a scan imaging protocol for a combination of input factors (e.g., scan protocol consisting of scan acquisition parameters (e.g. scan range, kV, etc.)) and scan reconstruction parameters (e.g. kernel, slice thickness, metal artifact reduction, etc.). The information of admission may include a mechanism of injury, demographics of the patient (e.g. age), clinical history (e.g. existing osteoporosis), etc.

The processor may use the machine/deep learning network to determine a scan imaging protocol based on at least one of patient information, mechanism of injury, optical camera images and a primary survey (e.g. Glasgow coma scale).

The processor may utilize the machine/deep learning network to extract the landmarks, anatomical labels and other geometrical information using a at least one of a 2D topogram(s), a low dose CT scan, a 2D camera, a 3D camera, “real time display” (RTD) images and an actual 3D scan performed by a CT scanner.

In an example embodiment, the processor may utilize the machine/deep learning network to extract the landmark coordinates, anatomical labels and other geometrical information on the anatomy, from one or more 2D topogram(s) (i.e., a scout image acquired for planning before the actual scan (CT, MR, etc.)). As topogram and 3D scans are in the same coordinate systems, anatomical information detected in 2D topogram(s) can be directly used in 3D tomographic scans, without any re-calculations. The advantage of such approach is a short processing time, since 2D topograms contain less data than a full 3D scan. The processor may use the machine/deep learning network to extract the landmark coordinates, anatomical labels and other geometrical information on the anatomy using conventional methods.

In another example embodiment, the processor may utilize the machine/deep learning network to extract the landmark coordinates, anatomical labels and other geometrical information on the anatomy using a 3D ultra low dose CT scan, which could be used as a preview and for planning of normal dose CT scans (thus fulfilling a similar function as a 2D topogram). The advantage of such approach is a higher precision due to the higher amount of information included in the 3D data. The processor may use the 3D ultra low dose CT scan to extract the landmark coordinates, anatomical labels and other geometrical information on the anatomy using conventional methods.

In another example embodiment, the processor may utilize the machine/deep learning network to extract the landmark coordinates, anatomical labels and other geometrical information on the anatomy using a 2D or 2D+time (video stream) camera image of the patient, acquired before the 3D scan. As for the topogram, anatomical information detected in 2D image(s) can be directly used in 3D tomographic scans, without any re-calculations. The machine/deep learning network may be trained with pairs of camera images and medical images (e.g., CT images) to perform landmark detection for internal landmarks (such as the position of the lungs, of the heart, etc.).

In another example embodiment, the processor may utilize the machine/deep learning network to extract the landmark coordinates, anatomical labels and other geometrical information on the anatomy using 3D (2D+depth) or 3D+time (video stream+depth) images acquired with camera devices like Microsoft Kinect™ camera. Anatomical information can be detected by the processor and used in a later step for processing of 3D scans. The depth information aids in obtaining a higher precision. The machine/deep learning network may be trained with pairs of 3D camera images and medical images (e.g., CT images) to perform landmark detection for internal landmarks (such as the position of the lungs, of the heart, etc.). By virtue of retrieving depth information, 3D cameras can see mechanical deformation due to breathing or heart beating that can be used to estimate the position of the respective organs.

In another example embodiment, the processor may utilize the machine/deep learning network to extract the landmark coordinates, anatomical labels and other geometrical information on the anatomy using the RTD images. RTD images are “preview” reconstructions, i.e., images reconstructed with a relatively low quality but with high speed. The RTD images may be displayed live during scanning so that a technician can see and monitor the ongoing scan. The machine/deep learning network may be trained with pairs of conventional CT images and RTD images to increase the speed of reconstruction while maintaining the quality of the image.

In another example embodiment, the processor may utilize the machine/deep learning network to extract the landmark coordinates, anatomical labels and other geometrical information on the anatomy using the actual 3D scan(s) (e.g. CT scan). In the case, where no topogram has been acquired (e.g. in order to save time), the anatomical information detection step can be performed on the same data that is going to be read.

In instances where the landmark coordinates, anatomical labels and other geometrical information on the anatomy are extracted before the actual 3D scan, the extracted landmark coordinates, anatomical labels and other geometrical information may be used for scan protocol selection and/or determining a CT reading algorithm.

For example, the extracted landmark coordinates, anatomical labels and other geometrical information patient illustrate an appearance that is indicative of specific injuries. This can also be used if clinical information/admission data is not available.

The processor may classify the specific injuries into known categories such as seat belt signs, gunshot wounds, pupil size, pupil dilation, for example. The machine/deep learning network may be trained with labeled images such as seat belt signs being bruises across the body and pupil sizes being an abnormality when compared to a set pupil size (e.g., an average size across the trained images).

The processor may then assign the categorized injury to a suspected condition. Possible suspected conditions corresponding to the categorized injury may be stored in a lookup table and the processor may select one of the possible suspected conditions based on the extracted landmark coordinates, anatomical labels and other geometrical information patient illustrate an appearance that is indicative of specific injuries. For example, dilated pupils may be assigned to a herniation, a seat belt injury may be assigned to thoracic injuries and lumps on the head may be assigned to positions of head injuries.

The assigned suspected condition may be used for scan protocol selection or determining a CT reading algorithm.

At S305, the processor uses the machine/deep learning network to segment the 3D image data into respective body regions/structures using the extracted landmarks, anatomical labels and other geometrical information. The segmentation may be done using known 3D segmentation techniques.

At S310, the processor uses the segmentations, the extracted landmarks, anatomical labels and other geometrical information to divide the 3D scan(s) into respective body regions/structures and to create a number of reconstructions. If prior to the CT scan, metallic objects have been introduced into the patient and detected in S300, a metal artifact reduction algorithm can be parameterized differently (e.g., to be more aggressive) by the processor. Moreover, the precise make, type/shape can be fed into a metallic artifact reduction algorithm as prior knowledge. Metallic objects may be detected in the topogram.

As will be described below in regards to data visualization, the processor may utilize the machine/deep learning network to select a format for a given body region and suspected conditions, to select kernels for the given body region and suspected conditions and to select a window for the given body region and suspected conditions.

In an example embodiment, the processor may utilize the machine/deep learning network to may divide acquired raw data (e.g. CT raw data before actual CT reconstruction) into different anatomical body regions and then perform dedicated reconstructions for the given body region in a customized manner. The processor may subdivide the acquired raw data based only on a z-coordinate of the anatomical landmarks. The processor may also reconstruct bony structures like spine with sharp kernel in such a way that spine centerline is perpendicular to the reconstructed images using the extracted landmarks, anatomical labels and other geometrical information.

In another example embodiment, the processor may utilize the machine/deep learning network to reconstruct the acquired raw data in a conventional manner and divide the reconstructed data, similarly as described above. For example, the processor may generate a whole body reconstructed CT scan and create dedicated subsets of the whole body reconstruction for separate anatomical structures (e.g., a head). The different subsets are created by the processor as a separate reconstruction with different visualization parameters. The visualization parameters include slice thickness, windowing and intensity projection (e.g., maximum intensity projection). The visualization parameters may be set by the processor using the machine/deep learning network. Moreover, reconstructions can be oriented in a different way (e.g. along the anatomical structures contained in the image). For example, for the head, the head reconstruction can be re-oriented to deliver images parallel to the skull base, based on the extracted landmarks, anatomical labels and other geometrical information.

The reconstructions can be created physically by the processor into DICOM images that can be sent to any medical device. Alternatively, the processor may generate the images virtually in the memory 103. The images may be used for visualization within dedicated software. By virtually generating the images, the time needed for transfer of reconstructed images will be reduced, as, e.g., only a whole body scan need to be transferred over the network, and the rest of the data is accessed directly in the memory 103.

At S315, the processor may utilize the machine/deep learning network to detect pathologies such as fractures, lesions or other injuries. The processor uses the machine/deep learning network to detect critical lesions faster than a human so that interventions can be administered earlier and it can be used to detect lesions that would be too subtle to see for a human such as a specific texture pattern or a very shallow contrast difference.

Based on the detected pathologies, the processor may perform organ and/or injury specific processes including automated processing of required information, detection of trauma-related findings, classification of findings into different subtypes, therapy decision making, therapy planning and automated incidental findings.

At S320, the processor generates a visualization as is described below.

### Data Visualization

As part of steps S310 and S315, the processor may utilize the machine/deep learning network to reformat an image, select kernels for reconstruction, select a window for a given body region (e.g., body region including extracted landmarks) and suspected conditions.

The machine/deep learning network may be trained with labeled images to determine formatting, kernels and windows for particular body regions and injuries in those regions. For example, the reformatting may be performed in a way that lesions are a desired visibility for a human reader. As an example, the processor may utilize the machine/deep learning network to reformat an image to change a plane where a laceration in a vessel is more visible than in a previous plane.

The processor may utilize the machine/deep learning network to select a kernel based on spatial resolution and noise. For example, the machine/deep learning network is trained to emphasize resolution for lesions with relatively smaller features and emphasize a kernel with better noise properties for lesions with a relatively weak contrast.

The processor may utilize the machine/deep learning network to select a window based on a detected lesions and injuries. For example, when a bone fracture is detected, the processor may select a bone window and when a brain injury is detected, the processor may select a soft tissue window.

In order to aid the technician's eye, graphical objects can be superimposed on findings in the CT image at S320, where geometrical properties of the superimposed objects (e.g. size, line-thickness, color, etc.) express the criticality of a certain finding.

For example, the processor may detect abnormal findings using the machine/deep learning network as described in S315. The processor may then retrieve from an external database and/or the memory 103 a criticality and assumed urgency of an intervention for the findings. The processor may then sort the findings according to criticality and assumed urgency of the intervention.

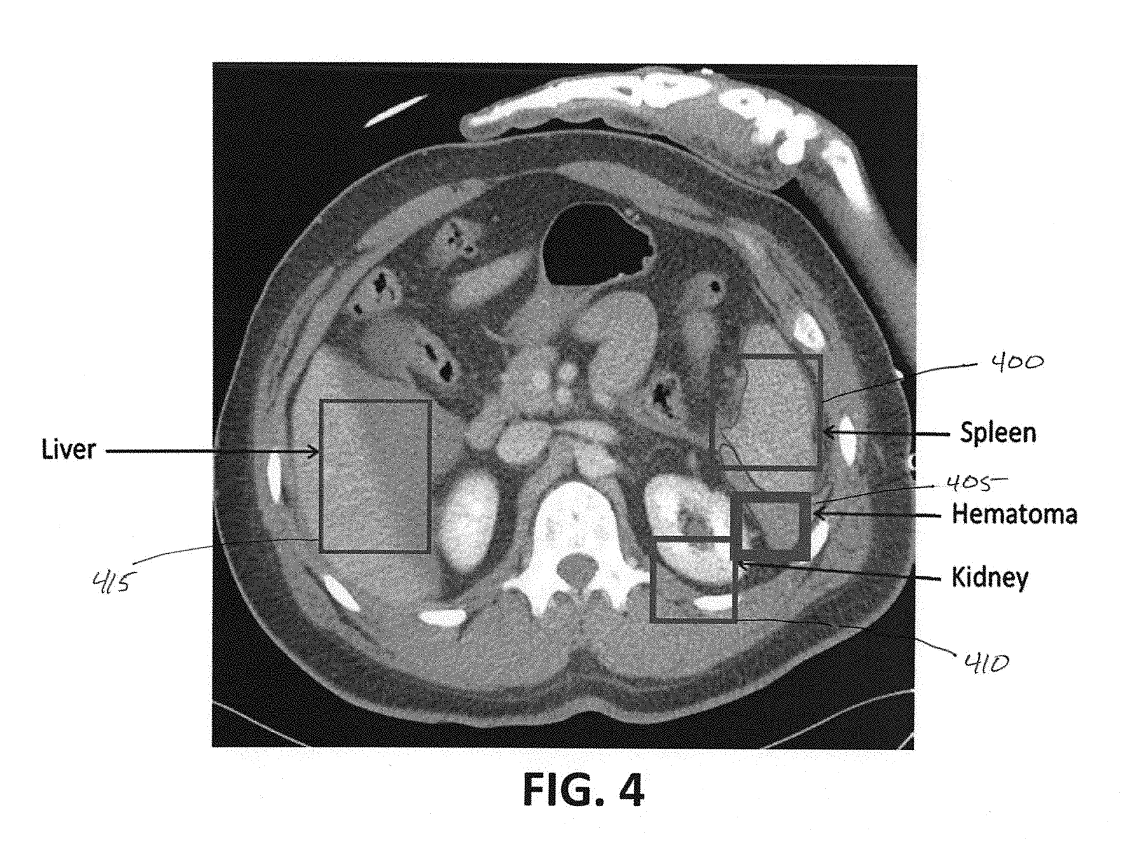

At S320, the processor assigns to each finding certain geometrical properties (e.g. size, line-thickness, color, etc.) which correlate with the order in the list of findings (i.e. more or less critical) and superimposes a rectangle on each finding (e.g. align with center of gravity for each finding). An example display is shown in FIG. 4.

As shown in FIG. 4, rectangles 400, 405, 410 and 415 are superimposed by the processor on findings related to a spleen injury, a hematoma, a kidney injury and a liver injury, respectively. Each of the rectangles 400, 405, 410 and 415 differs in the thickness of their border. The thickness (i.e., weight) represents the criticality. A thicker border represents a relatively more urgency and criticality. In FIG. 4, the rectangle 405 (corresponding to a hematoma) has the thickest border of the rectangles 400, 405, 410 and 415. Thus, the rectangle 405 surrounds the area of the image (i.e., a detected injury) have the highest criticality.

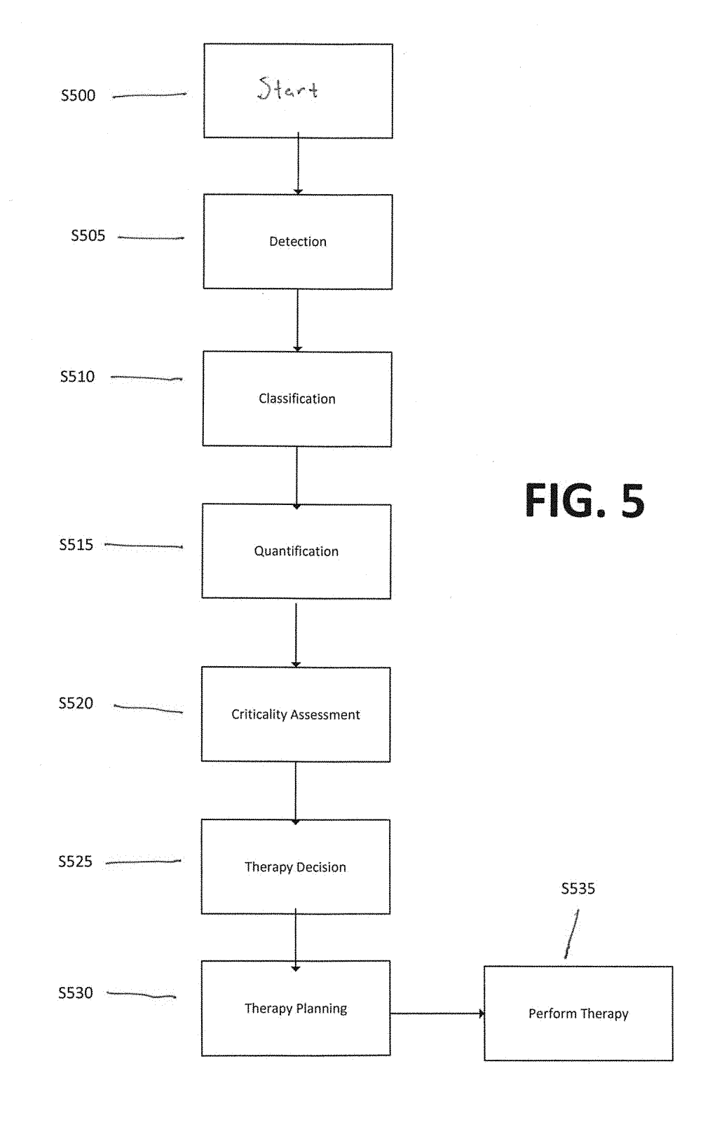

FIG. 5 illustrates a method of utilizing the machine/deep learning network for certain body regions, according to an example embodiment. The method of FIG. 5 and FIG. 3 are not exclusive and aspects of S300-S320 may be used in FIG. 5.

The method of FIG. 5 is initially described in general and then the method will be described with respect to certain body regions such as the head, face, spine, chest and abdomen.

At S500, the processor starts the process of utilizing the machine/deep learning network.

At S505, the processor utilizes the machine/deep learning network to detect injuries in the CT images and other additional scans (e.g., MRI). This may be done in the same manner as described in S320.

Using the detected injuries, the processor uses the machine/deep learning network to classify the injury at S510 by using a classification algorithm. The classification algorithm has a number of output categories matching the number of categories in the classification system. The algorithm works out probabilities that the target lesion could fall into any of these categories and assign it to the category with the highest probability. Probabilities are determined by the processor using the machine/deep learning network based on determining an overlap of the lesion with a number of features (either predefined or self-defined) that could relate to the shape, size, attenuation, texture, etc. The processor may classify the injury with an added shape illustrating the classified injury.

The processor then uses the machine/deep learning network to quantify the classified injury at S515. For example, the processor uses the machine/deep learning network to quantify a priori that is difficult for a radiologist to determine. By contrast, conventional systems and methods do not quantify a classified injury using machine/deep learning network.

At S520, the processor uses the machine/deep learning network to assess the criticality of the injury based on the quantification of the injury by comparing the quantified values against threshold values. For example, processor uses the machine/deep learning network to determine a risk of a patient undergoing hypovolemic shock by quantifying the loss of blood and determining whether the loss is higher than 20% of total blood volume. The processor uses the machine/deep learning network to determine a therapy based on the assessed criticality at S525 such as whether surgery should be performed in accordance with established clinical guidelines.

At S530, therapy planning is performed by the processor and then, at S535, the planned therapy is performed on the patient.

### Head

Using FIG. 5, the method of utilizing the machine/deep learning network for a head will be described.

At S505, the processor uses the machine/deep learning network to detect injuries in the CT images and other additional scans (e.g., MRI). For example, the processor may detect a diffuse axonal injury. Diffuse axonal injury is one of the major brain injuries that is hardest to conclusively diagnose on CT images. MRI scans are often used to clarify the diagnosis from the CT images. In order to detect diffuse axonal injury with more diagnostic confidence, the machine/deep learning network is trained with pairs of annotated CT and MRI images to determine correspondence between both images. Moreover, the machine/deep learning network may be trained to register both images, segment structures and highlight findings (e.g., superimpose geometrical shapes) in a CT image.

Using the detected injuries, the processor uses the machine/deep learning network to classify the injury at S510. For example, brain injuries can be classified by the processor according to at least one of shape, location of the injury and iodine content. The processor may classify the injury with an added shape illustrating the classified injury.

The processor then uses the machine/deep learning network to quantify the classified injury at S515.

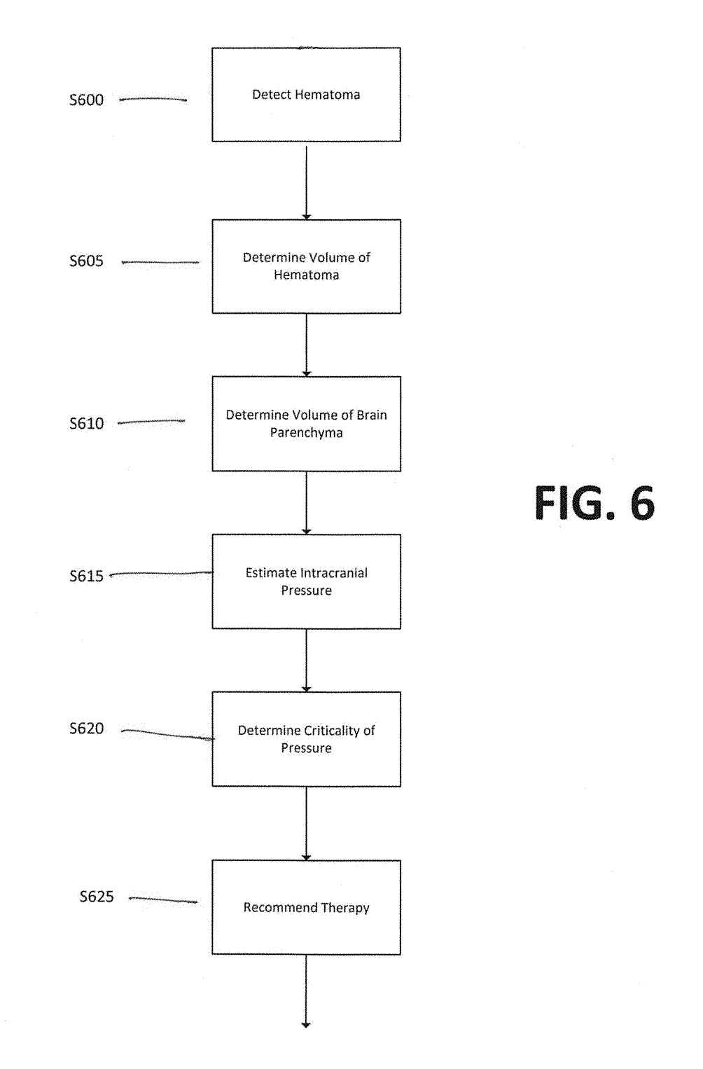

FIG. 6 illustrates an example embodiment of assessing the criticality of an injury in the head. More specifically, FIG. 6 illustrates a method of determining intracranial pressure due to a hematoma.

At 600, the processor uses the machine/deep learning network to detect a hematoma in the 3D CT data such as described with respect to S315. In addition, the processor may also determine a midline shift.

At 605, the processor uses the machine/deep learning network to determine volume of the hematoma by applying deep learning based 3D segmentation and performing a voxel count of the hematoma.

At 610, the processor uses the machine/deep learning network to determine a volume of a brain parenchyma by performing a distinction of non-parenchyma versus parenchyma with segmentation and performing a voxel count of the brain parenchyma.

At 615, the processor uses the machine/deep learning network to estimate an intracranial pressure by determining a volume inside the skull, determining a density and using the determined volume of the hematoma and the determined volume of the brain parenchyma.

At 620, the processor uses the machine/deep learning network to decide whether the intracranial pressure is critical by comparing the intracranial pressure to a determined threshold. The threshold may be determined based on empirical data.

At 625, the processor then uses the machine/deep learning to recommend a therapy such as non-operative, coagulation, Burr hole, craniotomy, now or delayed.

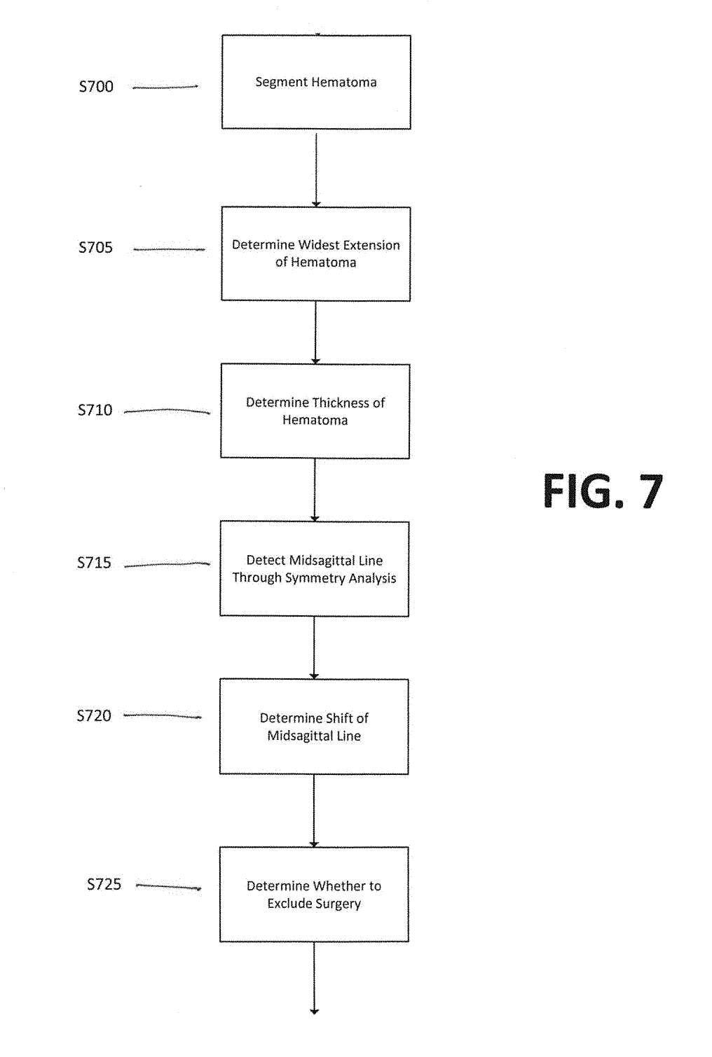

Referring back to FIG. 5, the processor then determines the therapy S525. An example embodiment of S525 is illustrated in FIG. 7.

At S700, the processor then uses the machine/deep learning network to segment the hematoma detected at S600 using deep learning based 3D segmentation.

At S705, the processor then uses the machine/deep learning network to determine a widest extension of the hematoma.

At S710, the processor uses the machine/deep learning network to determine thickness of the hematoma.

At S715, the processor then uses the machine/deep learning network to detect a midsagittal line through symmetry analysis using the detected landmarks.

At S720, the processor then uses the machine/deep learning network to determine a shift of the midsagittal line by detecting a deviation from symmetry or detecting a displacement of landmarks indicative of the midline.

The processor then determines whether to exclude surgery as a possible therapy based on the determinations performed in S705-S720. For example, the processor may exclude surgery for patients who exhibit an epidural hematoma (EDH) that is less than 30 mL, less than 15-mm thick, and have less than a 5-mm midline shift, without a focal neurological deficit and a Glasgow Comma Score (GCS) greater than 8 can be treated nonoperatively.

The processor may decide whether to perform surgery for a subdural hematoma by detecting basilar cisterns and determining whether compression or effacement is visible according to clinical guidelines.

Returning to FIG. 5, the processor uses the machine/deep learning network to plan the surgery or non-surgery at S530. Because the machine/deep learning network is used and the parameters are difficult to assess for humans, the evaluation can be made consistently. At S535, the therapy is performed.

### Face

With regards to a face of the patient, the processor uses the machine/deep learning network in automating a Le Fort fracture classification.

Le Fort fractures are fractures of the midface, which collectively involve separation of all or a portion of the midface from the skull base. In order to be separated from the skull base the pterygoid plates of the sphenoid bone need to be involved as these connect the midface to the sphenoid bone dorsally. The Le Fort classification system attempts to distinguish according to the plane of injury.

A Le Fort type I fracture includes a horizontal maxillary fracture, a separation of the teeth from the upper face fracture line passes through an alveolar ridge, a lateral nose and an inferior wall of a maxillary sinus.

A Le Fort type II fracture includes a pyramidal fracture, with the teeth at the pyramid base, and a nasofrontal suture at its apex fracture arch passes through posterior the alveolar ridge, lateral walls of maxillary sinuses, an inferior orbital rim and nasal bones.

A Le Fort type III fracture includes a craniofacial disjunction fracture line passing through the nasofrontal suture, a maxillo-frontal suture, an orbital wall, and a zygomatic arch/zygomaticofrontal suture.

The processor uses the machine/deep learning network to classify the Le Fort type fracture by acquiring 3D CT data of the head from the actual 3D CT scans and classifies the fracture into one of the three categories. The machine/deep learning network is trained with labeled training data using the description of the different Le Fort types above.

### Spine

Using FIG. 5, the method of utilizing the machine/deep learning network for a spine will be described.

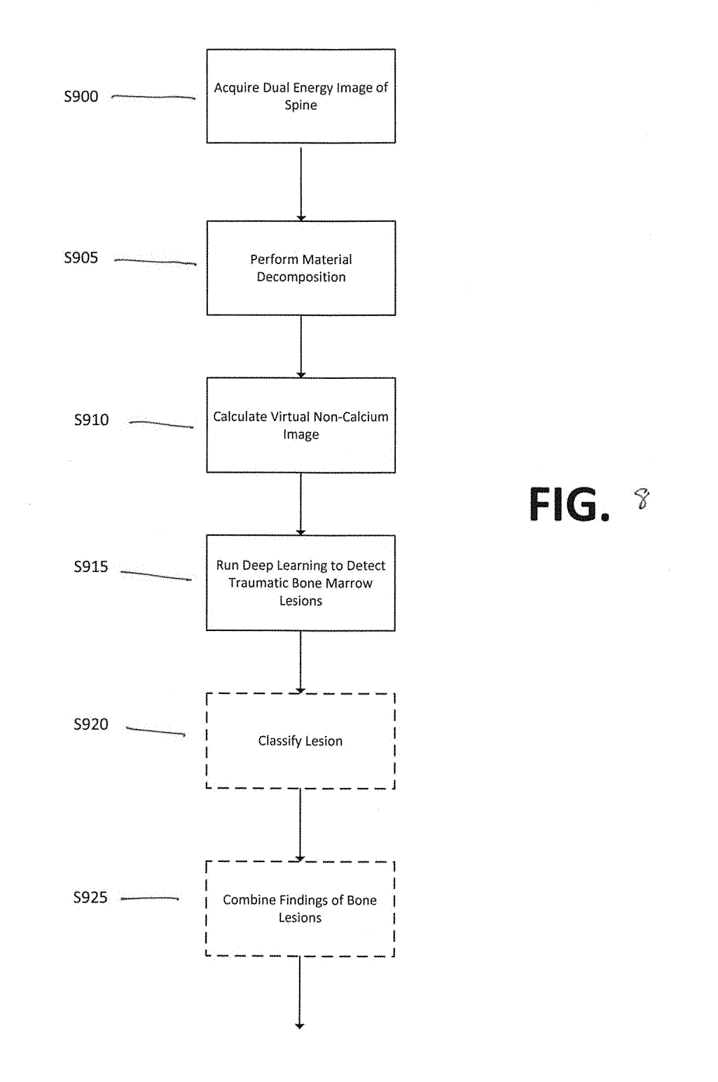

At S505, the processor uses the machine/deep learning network to detect injuries in the CT images and other additional scans (e.g., MRI). FIG. 8 illustrates an example embodiment of detecting traumatic bone marrow lesions in the spine.

At S900, the processor acquires a dual energy image of the spine from the CT scanner.

At S905, the processor performs a material decomposition on the dual energy image using any conventional algorithm. For example, the material decomposition may decompose the dual energy image to illustrate into three materials such as soft tissue, bone and iodine.

At S910, the processor calculates a virtual non-calcium image using the decomposed image data by removing the bone from the decomposed image using any conventional algorithm for generating a non-calcium image.

At S915, the processor uses the machine/deep learning network to detect traumatic bone marrow lesions in the virtual non-calcium image by performing local enhancements in the virtual non-calcium image at locations where bone was subtracted.

In addition, the processor may optionally classify a detected lesion into one of grades 1-4 at S920.

Moreover, the processor may combine findings of bone lesions that can be seen in conventional CT images at S925.

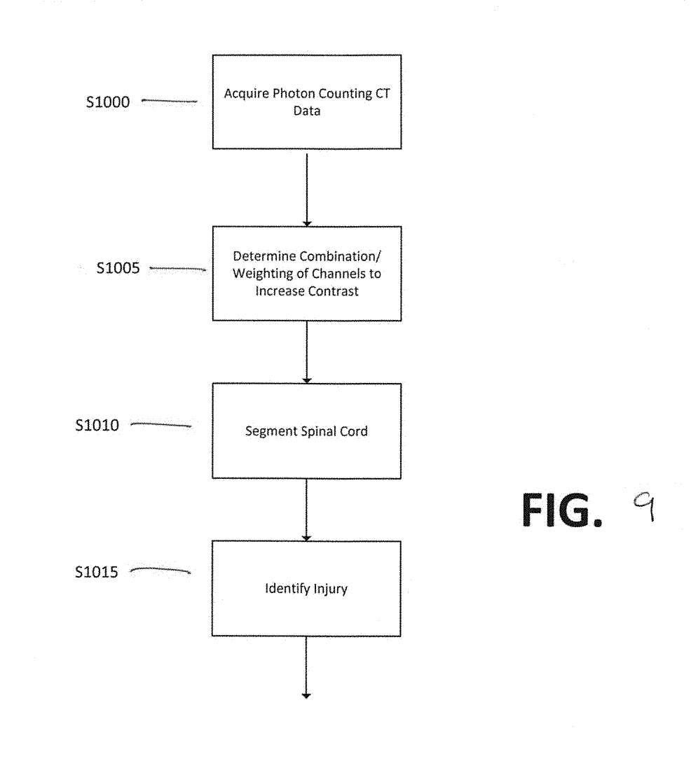

FIG. 9 illustrates an example embodiment of detecting a spinal cord in a patient.

At S1000, the processor acquires photon counting CT data with four spectral channels from the CT scanner (the CT scanner includes photon-counting detectors).

At S1005, the processor determines a combination and/or weighting of the spectral channels to increase contrast using a conventional algorithm.

At S1010, the processor uses the machine/deep learning network to identify injuries in the spine such as detect traumatic bone marrow lesions in the virtual non-calcium image spinal stenosis, cord transection, cord contusion, hemorrhage, disc herniation, and cord edema.

Returning to FIG. 5, using the detected injuries, the processor uses the machine/deep learning network to classify the injury at S510.

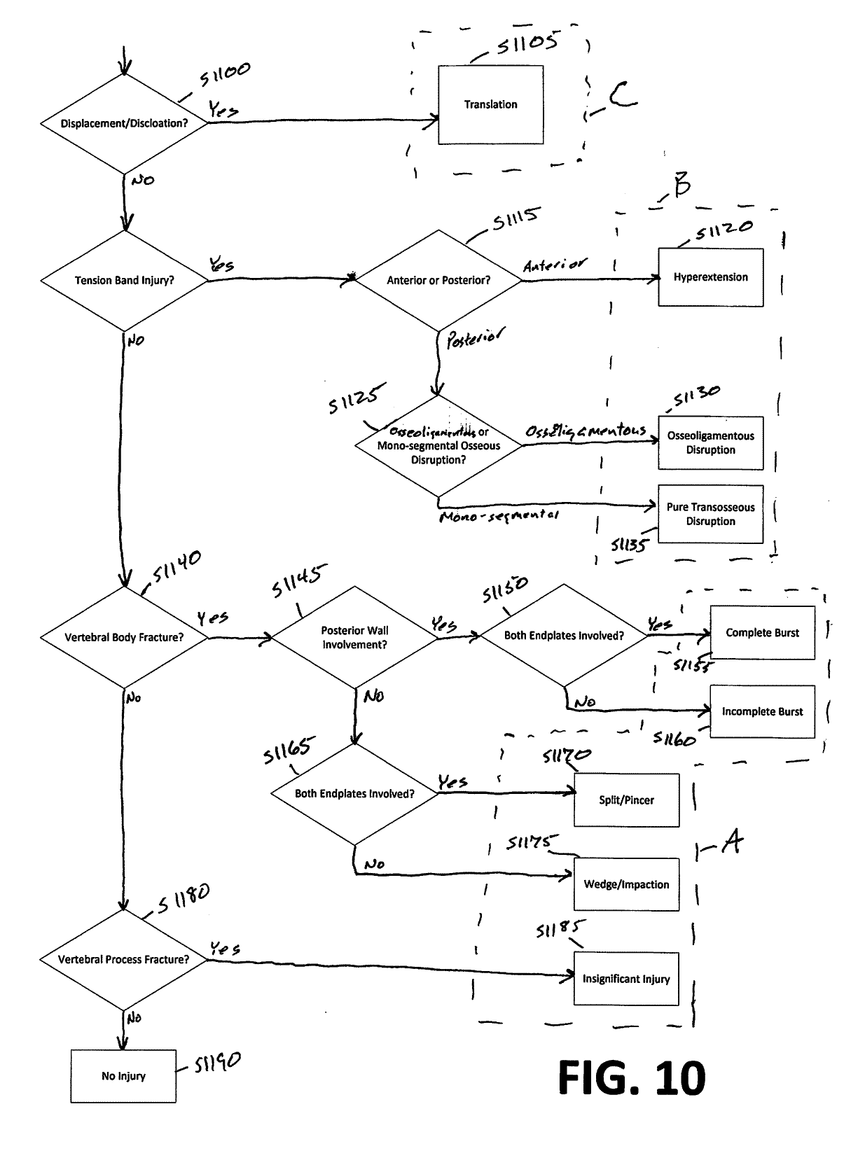

FIG. 10 illustrates an example embodiment of classifying a spinal fracture.

As shown in FIG. 10, spinal fractures may be classified into Types A, B and C. Type A is compression fractures, Type B is distraction fractures and Type C is displacement or translation fractures.

At S1100, the processor determines whether there is a displacement or dislocation in the CT image data.

If there is a displacement or dislocation, the processor classifies the injury as a translation injury at S1105.

If the processor determines no displacement or dislocation exists, the processor determines whether there is a tension band injury at S1110. If the processor determines there is a tension band injury, the processor determines whether the injury is anterior or posterior at S1115. If the injury is determined to be anterior, the processor classifies the injury at hyperextension at S1120. If the injury is determined to be posterior, the processor determines a disruption at S1125. When the processor determines the disruption to be an osseoligamentous disruption, the processor classifies the injury as the osseoligamentous disruption at S1130. When the processor determines the disruption to be a mono-segmental osseous disruption, the processor classifies the injury as a pure transosseous disruption at S1135. Hypertension, osseoligamentous disruption and pure transosseous disruption are considered type B injuries as shown in FIG. 10.

If the processor determines there is no tension band injury at S1110, the processor proceeds to S1140 and determines whether there is a vertebral body fracture. If the processor determines in the affirmative, the processor determines whether there is posterior wall involvement at S1145. If the processor determines there is posterior wall involvement, the processor determines whether both endplates are involved at S1150. The processor classifies the injury as a complete burst at S1155 if both endplates are involved and classifies the injury as an incomplete burst at S1160 if both endplates are not involved. If the processor determines that there is no posterior wall involvement at S1145, the processor determines whether both endplates are involved at S1165. The processor classifies the injury as a split/pincer at S1170 if both endplates are involved and classifies the injury as a wedge/impaction at S1175 if both endplates are not involved.

If the processor determines there is no vertebral body fracture at S1140, the processor determines whether there is a vertebral process fracture at S1180. If the processor determines there is a vertebral process fracture at S1180, the processor classifies the injury as an insignificant injury at S1185. If the processor determines there is not a vertebral process fracture at S1180, the processor determines there is no injury at S1190.

Complete burst, incomplete burst, split/pincer, wedge/impaction and insignificant injury are considered type A injuries, as shown in FIG. 10.

Returning to FIG. 5, the processor then uses the machine/deep learning network to quantify the classified injury at S515.

At S520, the processor uses the machine/deep learning network to assess the criticality of the spinal injury. For example, the processor may use the machine/deep learning network to assess the stability of a spine injury by applying virtual forces that emulate the patient standing and/or sitting.

For every vertebrae, the processor may detect a position, an angle and a distance to adjacent vertebrae. The processor may detect fractures based on the applied virtual forces, retrieve mechanical characteristics of the bones from a database, and apply virtual forces using the machine/deep learning network to emulate the sitting and/or standing of the patient. The machine/deep learning network is trained using synthetic training data acquired through the use of finite element simulation, thus enabling the processor to emulate the sitting and/or standing of the patient.

Based on the results of the sitting and/or standing emulation, the processor decides the risk of fracture/stability.

The processor then uses the assessed criticality to determine the therapy and plan the therapy at S525 and S530.

### Chest

Using FIG. 5, the method of utilizing the machine/deep learning network for a chest will be described.

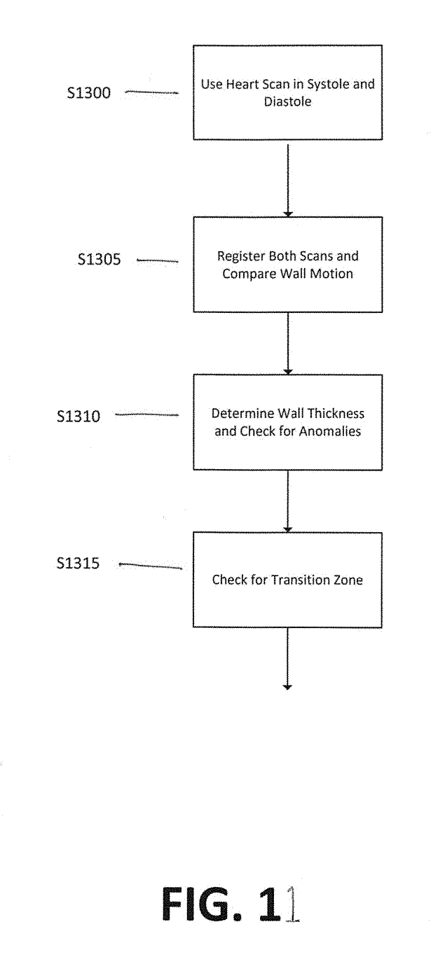

At S505, the processor uses the machine/deep learning network to detect injuries in the CT images and other additional scans (e.g., MRI). FIG. 11 illustrates an example embodiment of detecting a cardiac contusion.

At S1300, the processor acquires a CT image data of the heard in systole and diastole.

At S1305, the processor registers both scans (systole and diastole) and compares wall motion of the heart with already stored entries in a database. The processor determines the wall thickness of the heart of the patient and check for anomalies at S1310. To distinguish from myocardial infarction, the processor uses the machine/deep learning network to determine whether the tissue shows a transition zone (infraction) or is more confined and has distinct edges (contusion) at S1315.

Returning to FIG. 5, the processor uses the machine/deep learning network to classify the detected heart injury. For example, the processor uses the machine/deep learning network to classify aortic dissections using the Stanford and/or DeBakey classification. The processor uses the machine/deep learning network to detect the aorta, detect a dissection, detect a brachiocephalic vessel, determine whether dissection is before or beyond brachiocephalic vessels and classify the dissection into type a or b (for Stanford) and/or type i, ii or iii (for DeBakey).

At S515, the processor uses the machine/deep learning network to quantify the heart injury.

At S520, the heart assesses the criticality of the heart injury. For example, the processor uses the machine/deep learning network to detect detached bone structures, determine a quantity, size, position and sharpness for the detached bone structures, decide whether lung function is compromised and decide whether surgery is required. The processor uses the machine/deep learning network to decide whether surgery is required by comparing the determined quantity, size, position and sharpness of detached bone structures and lung functionality to set criteria. The set criteria may be determined based on empirical data.

The processor then uses the assessed criticality to determine the therapy and plan the therapy at S525 and S530.

### Abdomen

Using FIG. 5, the method of utilizing the machine/deep learning network for an abdomen will be described.

At S505, the processor utilizes the machine/deep learning network to detect a spleen injury in accordance with the automated AAST Spleen Injury Scale based on CT images.

At S505, the processor uses the machine/deep learning network to detect the spleen, a liver and a kidney on the CT image.

The processor then uses the machine/deep learning network to detect a hematoma on the spleen, liver and/or kidney after segmenting the spleen, liver and kidney.

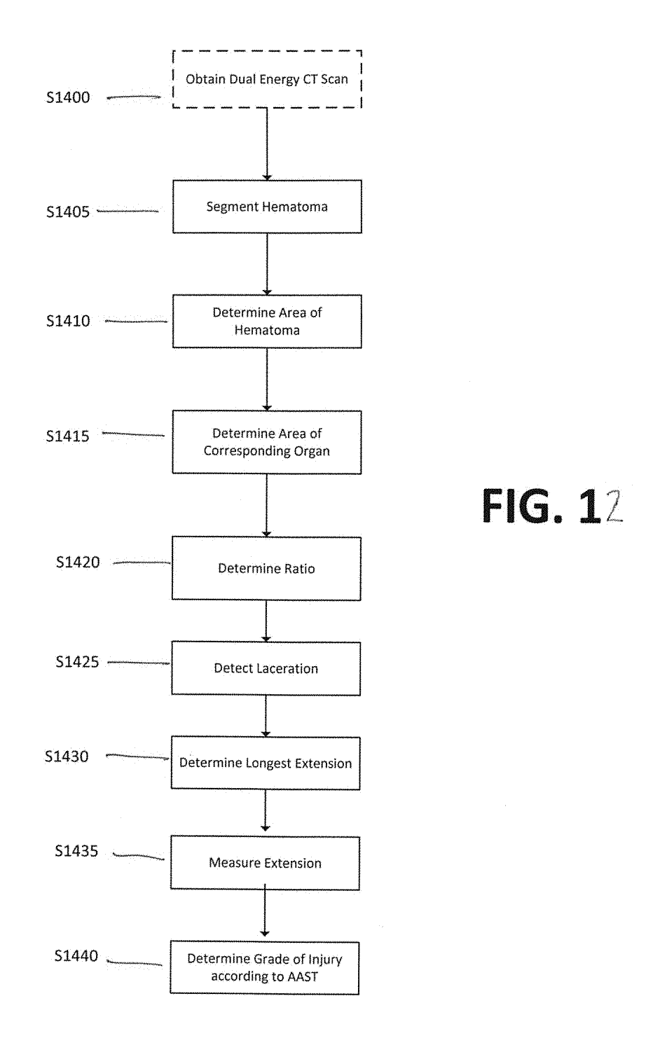

FIG. 12 illustrates an example embodiment of the detection, classification, quantification and criticality assessment of a hematoma on the spleen, liver or kidney. The processor uses the machine/deep learning network to perform the steps shown in FIG. 12.

At S1400, the processor may optionally obtain a dual energy CT scan to aid delineation of the organ and hematoma as well as differential of hematoma versus extravasation of contrast material.

At S1405, the processor segments the hematoma using conventional segmentation algorithms (e.g., watershed, thresholding, region growing, graph cuts, model based).

At S1410, the processor determines and area of the hematoma and determines area of the corresponding organ at S1415.

At S1420, the processor determines a ratio of the area of the hematoma to the area of the corresponding organ.

At S1425, the processor detects laceration on spleen, liver and kidney.

At S1430, the processor finds a longest extension of the laceration and measures the extension at S1435.

At S1440, the processor determines a grade of the corresponding solid organ injury according to AAST Spleen Injury Scale.

Return to FIG. 5, a therapy decision may be made. For example, a solid organ (e.g., spleen, kidney or liver) can be tracked across multiple follow-up CT scans and different emergency intervention may be determined such as embolization, laparoscopy, or explorative surgery. For example, the process may register current and prior images using conventional registration algorithms, detect an injury in the prior image and follow up using the machine/deep learning to quantify injuries and to determine changes in size, density, area, volume, shape. The processor may then classify injury progression into one of many therapeutic options.

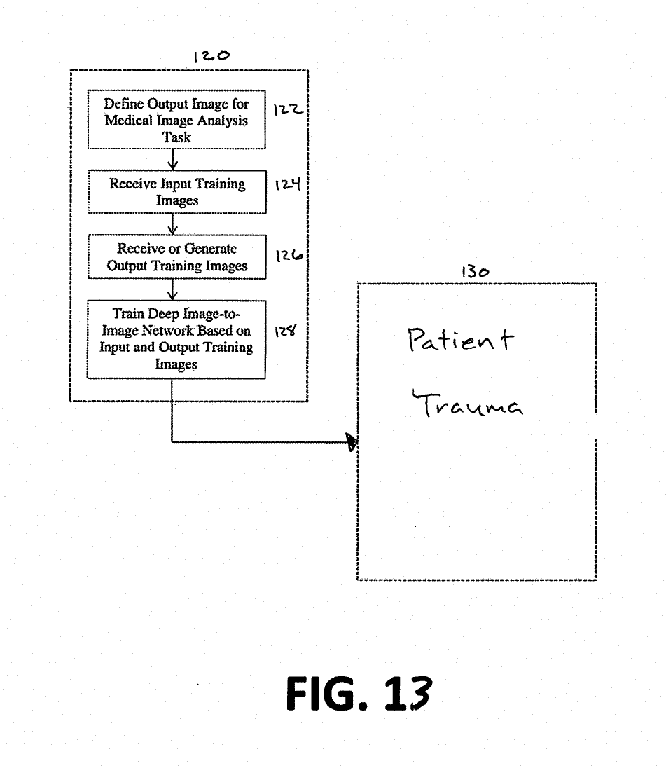

FIG. 13 illustrates a method for training the machine/deep learning network according to an example embodiment. The method of FIG. 13 includes a training stage 120 and an implementation stage 130. The training stage 120, which includes steps 122-128, is performed off-line to train the machine/deep learning network for a particular medical image analysis task such as patient trauma, as described above with respect to FIGS. 1-11. The testing stage 130, performs the trauma analysis using the machine/deep learning network resulting from the training stage 120. Once the machine/deep learning network is trained in the training stage 120, the testing stage 130 can be repeated for each newly received patient to perform the medical image analysis task on each newly received input medical image(s) using the trained machine/deep learning network.

At step 122, an output image is defined for the medical image analysis task. The machine/deep learning framework described herein utilizes an image-to-image framework in which an input medical image or multiple input medical images is/are mapped to an output image that provides the result of a particular medical image analysis task. In the machine/deep learning framework, the input is an image I or a set of images I₁, I₂, . . . , I_{N} and the output will be an image J or a set of images J₁, J₂, . . . , J_{M}. An image I includes a set of pixels (for a 2D image) or voxels (for a 3D image) that form a rectangular lattice Ω={x} (x is a 2D vector for a 2D image and a 3D vector for a 3D image) and defines a mapping function from the lattice to a desired set, i.e., {I(x)εR; xεΩ} for a gray-value image or {I(x)εR³; xεΩ} for a color image. If a set of images are used as the input, then they share the same lattice Ω; that is, they have the same size. For the output image J, its size is often the same as that of the input image I, though different lattice sizes can be handled too as long as there is a defined correspondence between the lattice of the input image and the lattice of the output image. As used herein, unless otherwise specified, a set of images I₁, I₂, . . . , I_{N} will be treated as one image with multiple channels, that is {I(x)εR^{N}; xεΩ} for N gray images or {I(x)εR³ xεΩ} for N color images.

The machine/deep learning framework can be used to formulate many different medical image analysis problems as those described above with respect to FIGS. 1-11. In order to use the machine/deep learning framework to perform a particular medical image analysis task, an output image is defined for the particular medical image analysis task. The solutions/results for many image analysis tasks are often not images. For example, anatomical landmark detection tasks typically provide coordinates of a landmark location in the input image and anatomy detection tasks typically provide a pose (e.g., position, orientation, and scale) of a bounding box surrounding an anatomical object of interest in the input image. According to an example embodiment, an output image is defined for a particular medical image analysis task that provides the result of that medical image analysis task in the form of an image. In one possible implementation, the output image for a target medical image analysis task can be automatically defined, for example by selecting a stored predetermined output image format corresponding to the target medical image analysis task. In another possible implementation, user input can be received corresponding to an output image format defined by a user for a target medical image analysis task. Examples of output image definitions for various medical image analysis tasks are described below.

For landmark detection in an input medical image, given an input medical image I, the task is to provide the exact location(s) of a single landmark or multiple landmarks of interest {x₁, I=1, 2, . . . }. In one implementation, the output image J can be defined as:

J(x)=ΣₗΣᵢ*g(|x−x₁|;σ), (1)

This results in a mask image in which pixel locations of the landmark l have a value of 1, and all other pixel locations have a value of zero. In an alternative implementation, the output image for a landmark detection task can be defined as an image with a Gaussian-like circle (for 2D image) or ball (for 3D image) surrounding each landmark. Such an output image can be defined as:

J(x)=Σₗτᵢ*g(|x−x₁|;σ) (2)

where g(t) is a Gaussian function with support σ and |x−x₁| measures the distance from the pixel x to the 1^{th} landmark.

For anatomy detection, given an input image I, the task is to find the exact bounding box of an anatomy of interest (e.g., organ, bone structure, or other anatomical object of interest). The bounding box B(θ) can be parameterized by θ. For example, for an axis-aligned box, θ=[x_{c},s], where x_{c} is the center of the box and s is the size of the box. For a non-axis-aligned box, θ can include position, orientation, and scale parameters. The output image J can be defined as:

J(x)=1 if xεB(θ); otherwise 0. (3)

This results in a binary mask with pixels (or voxels) equal to 1 within the bounding box and equal 0 at all other pixel locations. Similarly, this definition can be extended to cope with multiple instances of a single anatomy and/or multiple detected anatomies.

In lesion detection and segmentation, given an input image I, the tasks are to detect and segment one or multiple lesions. The output image J for lesion detection and segmentation can be defined as described above for the anatomy detection and segmentation tasks. To handle lesion characterization, the output image J can be defined by further assigning new labels in the multi-label mask function (Eq. (4)) or the Gaussian band (Eq. (5)) so that fine-grained characterization labels can be captured in the output image.

For image denoising of an input medical image. Given an input image I, the image denoising task generates an output image J in which the noise is reduced.

For cross-modality image registration, given a pair of input images {I₁,I₂}, the image registration task finds a deformation field d(x) such that I₁(x) and I₂(x−d(x)) are in correspondence. In an advantageous implementation, the output image J(x) is exactly the deformation field, J(x)=d(x).

For quantitative parametric mapping, given a set of input images {I₁, . . . , Iₙ} and a pointwise generative model {I₁, . . . , Iₙ}(X)=F(J₁, . . . Jₘ.)(X), a parametric mapping task aims to recover the quantitative parameters that generated the input images. An examples of quantitative mapping tasks includes material decomposition from spectral CT.

It is to be understood, that for any medical image analysis task, as long as an output image can be defined for that medical image analysis task that provides the results of that medical image analysis task, the medical image analysis task can be regarded as a machine/deep learning problem and performed using the method of FIG. 13.

Returning to FIG. 13, at step 124, input training images are received. The input training images are medical images acquired using any type of medical imaging modality, such as computed tomography (CT), magnetic resonance (MR), DynaCT, ultrasound, x-ray, positron emission tomography (PET), etc. The input training images correspond to a particular medical image analysis task for which the machine/deep learning network is to be trained. Depending on the particular medical image analysis task for which the machine/deep learning network is to be trained, each input training image for training the machine/deep learning network can be an individual medical image or a set of multiple medical images. The input training images can be received by loading a number of previously stored medical images from a database of medical images.

At step 126, output training images corresponding to the input training images are received or generated. The machine/deep learning network trained for the particular medical image analysis task is trained based on paired input and output training samples. Accordingly for each input training image (or set of input training images), a corresponding output training image is received or generated. The output images for various medical image analysis tasks are defined as described above in step 122. In some embodiments, the output images corresponding to the input training images may be existing images that are stored in a database. In this case, the output training images are received by loading the previously stored output image corresponding to each input training image. In this case, the output training images may be received at the same time as the input training images are received. For example, for the image denoising task, a previously stored reduced noise medical image corresponding to each input training image may be received. For the quantitative parametric mapping task, for each set of input training images, a previously acquired set of quantitative parameters can be received. For landmark detection, anatomy detection, anatomy segmentation, and lesion detection, segmentation and characterization tasks, if previously stored output images (as defined above) exist for the input training images, the previously stored output images can be received.

In other embodiments, output training images can be generated automatically or semi-automatically from the received input training images. For example, for landmark detection, anatomy detection, anatomy segmentation, and lesion detection, segmentation and characterization tasks, the received input training images may include annotated detection/segmentation/characterization results or manual annotations of landmark/anatomy/lesion locations, boundaries, and/or characterizations may be received from a user via a user input device (e.g., mouse, touchscreen, etc.). The output training images can then be generated by automatically generating a mask images or Gaussian-like circle/band image as described above for each input training image based on the annotations in each input training image. It is also possible, that the locations, boundaries, and/or characterizations in the training input images be determined using an existing automatic or semi-automatic detection/segmentation/characterization algorithm and then used as basis for automatically generating the corresponding output training images. For the image denoising task, if no reduced noise images corresponding to the input training images are already stored, an existing filtering or denoising algorithm can be applied to the input training images to generate the output training images. For the cross-modality image registration task, the output training images can be generated by registering each input training image pair using an existing image registration algorithm to generate a deformation field for each input training image pair. For the quantitative parametric mapping task, the output training image can be generated by applying an existing parametric mapping algorithm to each set of input training images to calculate a corresponding set of quantitative parameters for each set of input training images.

At step 108, the machine/deep learning network is trained for a particular medical image analysis task based on the input and output training images. During training, assuming the availability of paired training datasets {(Iₙ(x),Jₙ(x)); n=1, 2, . . . }, following the maximum likelihood principle, the goal of the training is to maximize a likelihood P with respect to a modeling parameter θ. The training learns the modeling parameter θ that maximizes the likelihood P. During the testing (or estimation/inference) stage (130 of FIG. 13), given an newly received input image I(x), an output image is generated that maximizes the likelihood P(J(x)I|(x); θ) with the parameter θ fixed as the parameter learned during training. An example of training the machine/deep learning network is further described in U.S. Pat. No. 9,760,807, the entire contents of which are hereby incorporated by reference.

### User Interface

As described above, anatomical information is determined within the coordinate system of 3D scans (e.g., CT scans). The anatomical information can be used for various purposes which are described below. The processor 102 may perform the functions described below by executing computer-readable instructions stored in the memory 103 to generate the UI. Moreover, the diagnostic workstations 15.k may be configured to perform the functions as well.

The UI may be considered part of reading software used to read the generated CT scans.

The UI may include a navigation element to navigate automatically to a given anatomical region. The processor may then create an anatomical region, virtually or physically, using the segmentation and reconstruction described above. Moreover, the UI may include a layout supporting answering of dedicated clinical questions (e.g. bone fractures or bleeding), irrespective of a given body region.

Within a given anatomical region or within clinical question, the UI may display data for reading for the anatomical region. For example, the UI may display RTD images along with the images from the CT scan. Conventional, RTD images are only displayed live during scanning at the scanner console and they are not used during reading. However, in trauma practice, a radiologist already looks at RTD images in order to spot life-threatening injuries as fast as possible. In order to support that, the UI displays and uses the RTD images within the reading software.

The UI may also display reconstructed images for different body parts (physical or virtual reconstructions) within dedicated layouts for reading for a given body part.

In addition, in order to save the time needed for transferring different reconstructions for various kernels to the workstations 15.k, instead of storing and transferring data for all possible kernels, “virtual kernels” can be created on the fly.

A dedicated UI element can be stored for each segment, thereby allowing a user to dynamically switch from one kernel to another. In this case, the system can also consider that data from one reconstruction is included in multiple segments (e.g. axial, sagittal and coronal views) and can automatically switch between kernels for all of associated views.

In some example embodiments, the system can make use of functional imaging data which either has been calculated on the image acquisition device (CT scanner) or it can be calculated on the fly within the trauma reading software. For example, when using dual energy data, the system provides dedicated layouts for e.g. bleeding detection the system can automatically calculate and display iodine maps for this purpose.

As preparing the data for display within a given segment or layout might need some seconds of preparation time, the system may display a status of loading/processing on or close to the navigational elements. Also, a status of general availability of the data for a given body region can be displayed (e.g., the head might not be available in the acquired images).

Within a given anatomical region, the UI includes dedicated tools for visualization and processing of the data such that the data can be displayed in segments and reformatted based on anatomical information.

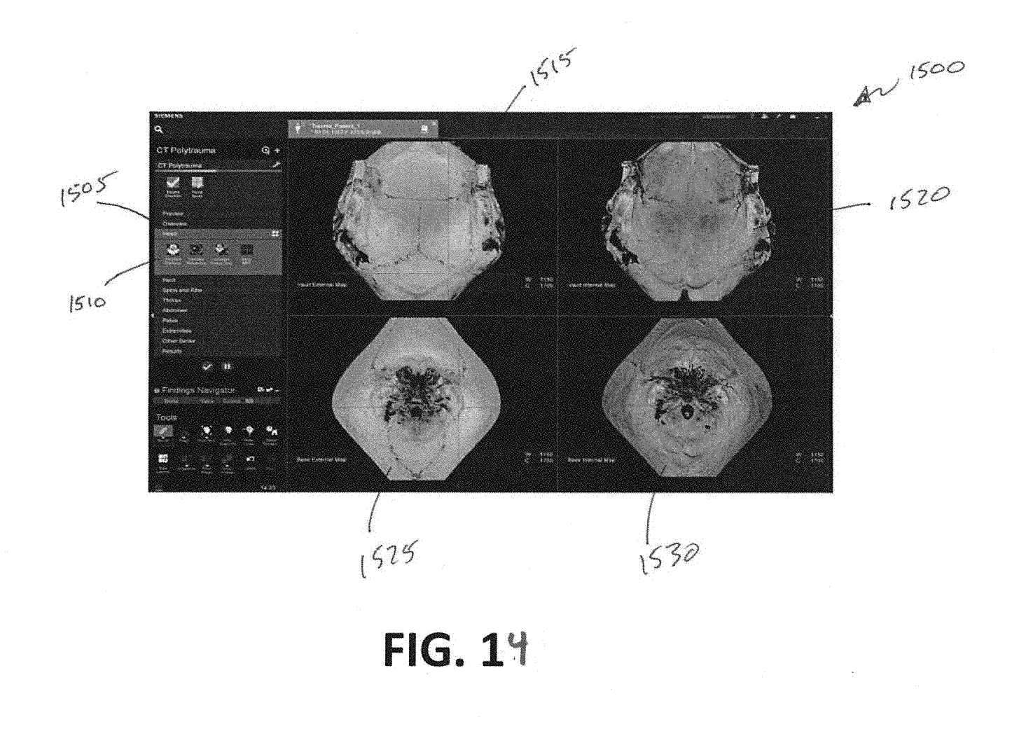

The UI may maintain the orientation of the data for a given body region. For example, an example embodiment of a UI is illustrated in FIG. 14. As shown, a UI includes a list of navigation elements 1505 including a navigation element for a head of the patient 1510. Upon the navigation element 1510 being selected (e.g., a user clicks on a navigation element “head”) and the processor executes software to display images 1515, 1520, 1525 and 1530 of the head in the segment.

As default, the system may display a middle image of a given anatomical region. However, example embodiments are not limited thereto and other anatomical positions within the region can be displayed by default. The user can then scroll up and down in the segments, from the top to the bottom of the head.

Moreover, the system may rotate and translate the image data using the anatomical information of the patient. For example, the system may present symmetrical views of a patient's brain if the patient has his head leaned to a direction during the scan.

The system may re-process the data and a display of a given anatomical structure is generated. For example, a “rib unfolding view” can be presented to a user. Moreover, extracting skull structures and displaying a flattened view of the skull to the user may be performed by the system as described in U.S. Pat. No. 8,705,830, the entire contents of which are hereby incorporated by reference.

For each body region, the system may provide dedicated tools for reading. Such context-sensitive tools can help to maintain overview of the UI and can speed the reading process. For example, the system may provide tools for inspecting body lesions for a spine. For vessel views, the system may provide tools for measuring vessel stenosis.

While the user creates findings and/or reports on given findings, the system can use this information to support the user. For example, the user can create a marker in a vertebra and the system automatically places a respective vertebra label in the marker. In addition, image filters, like slab thickness, MIP, MIP thin, windowing presets, are available within the segments.

The system permits a user to configure the dedicated tools and how the data is displayed (e.g., the visualization of each body region). In this context, the configuration can be either static or the system can learn dynamically from the usage (e.g., by machine learning, the system can learn, which data is preferably displayed by the user in which segments, which visualization presets, like kernel or windowing are applied, etc.). Also, if the user re-orientates images, the system can learn from this and present images re-oriented accordingly next time.

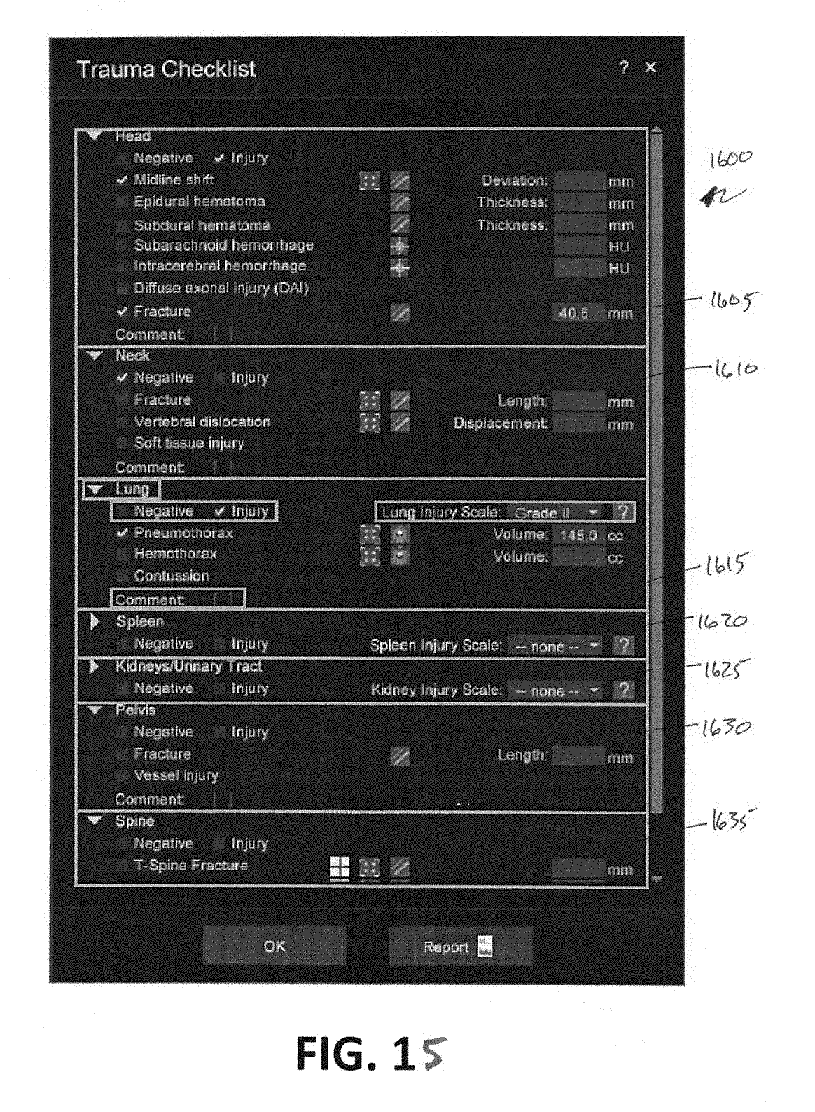

FIG. 15 illustrates an example embodiment of an interactive checklist generated by the system. As shown in FIG. 15, a checklist 1600 includes groups 1605, 1610, 1615, 1620, 1625, 1630 and 1635 divided according to body region (e.g., head, neck, lung, spleen, kidneys, pelvis and spine).

The system may expand/collapse the groups 1605, 1610, 1615, 1620, 1625, 1630 and 1635 based on an input from the user. An entire group may be marked as being injury, the severity of the injury may be assessed using an injury scale and the user may provide text comments.

Elements in the checklist can allow navigation to given body regions and elements can include dedicated tools for measuring/analyzing various pathologies. On activation of such a tool, the system can provide an optimal view for analysis.

For example, if Jefferson's fracture is on the checklist the system can automatically navigate to C1 vertebra and provide reformatted view through the anterior and posterior arches on activation of a dedicated position in the checklist. At the same time, a measuring tool can be activated so that the user (radiologist) can make a diagnosis/measure if such fracture occurred or not.

Upon receiving an indication that the user has selected a given item in the checklist, the system can present pre-analyzed structure/pathology such as detected and pre-measured Jefferson fracture.

The data filled into the checklist by radiologist or automatically by the system can later be transferred over a defined communication channel (e.g., HL7 (Health Level Seven)) to the final report (e.g. being finalized on another system like radiology information system (RIS)).

For trauma reading, first and second reads may be performed. Within the first pass, the most life-threatening injuries are in focus, whereas during the second reading pass, all of aspects including incidental findings are read and reported by the radiologist.

Distinguishing if first or second read is currently performed can be taken explicitly by the user by some UI element, automatically based on the time between the scan and reading (short time means first read, longer time means second read) or based on the information if this case has already been opened with reading software. For the case that the patient has been opened with the same software, some information shall be stored within first read. For the case that the patient has been opened with a different software, a dedicated communication protocol is used. Depending on first or second read, different options (tools, visualization, etc.) for different body parts can be provided and e.g. a different checklist can be shown to the user (one checklist for life-threatening injuries, and one, more holistic list, for final, second read). Also, all findings created during the first read need to be stored and available for the second read so that radiologist does not need to repeat his or her work.

### Trajectory

For wounds created by objects penetrating the body, radiologists usually try to follow the trajectory of the objects within the images manually. They find the entry (and in some cases the exit point) and by scrolling, rotating, translating and zooming the images they try to follow the penetration trajectory while assessing the impact of the wound on the objects along the trajectory. However, sometimes the injuries are not immediately visible, e.g. if a foreign objects goes through a part of the body where no dense tissue is present, e.g. within abdomen.

The system shown in FIGS. 1 and 2A help analyze images along the trajectory of a penetrating objects. In one example embodiment, a user can provide/mark entry and exit points and other internal points within the body. In another example embodiment, the system can automatically find one or more of those points along the trajectory of a penetrating object using the machine/deep learning network. The detection can be conducted by machine/deep learning network, based on a set of previously annotated data.

Based on the entry and exit points and other internal points within the body, the system may determine the trajectory path.

In one example embodiment, the system calculates a line/polyline/interpolated curve or other geometrical figure connecting the entry and exit points and other internal points within the body.

In another example embodiment, the system calculates the trajectory of the penetrating object based on at least one of image information provided by the user and traces of the object detected in the images.

In another example embodiment, the system calculated the trajectory based on a model, which may be a biomechanical simulation model considering type of object (bullet, knife, etc.) and the organs/structures along the path.

A dedicated visualization (e.g. rectangles, circles, markers, etc.) can be taken for visualization of the entry and exit points. The system takes the geometry of the trajectory, and displays the trajectory as an overlay over the medical images. The trajectory overlay (including entry and exit points) can be turned on or off by the user in order to see the anatomy below. As a special visualization a curved planar reformatting (CPR) or straightened CPR of the trajectory can be displayed. The user can then rotate the CPR around the trajectory centerline or scroll the CPR forth and back. Such visualizations help to analyze the whole path of the penetrating object with less user interaction and will help to ensure that the radiologist followed the whole penetration path during the reading.

The system can provide a way to automatically or semi-automatically navigate along the trajectory line. For example, within a dedicated layout, in one segment, the software can provide a view perpendicular to the trajectory, while in other segments e.g. a CPR of the trajectory is displayed. The user can navigate along the trajectory path in one or other direction by mouse or keyboard interaction. Alternatively, the software flies along the trajectory automatically with a given speed (that could also be controlled by the user). Also a combination of both automatic and semi-automatic navigation is possible.

Example embodiments being thus described, it will be obvious that the same may be varied in many ways. Such variations are not to be regarded as a departure from the spirit and scope of example embodiments, and all such modifications as would be obvious to one skilled in the art are intended to be included within the scope of the claims.

1. A method for assessing a patient, the method comprising:
determining scan parameters of the patient using machine learning;
scanning the patient using the determined scan parameters to generate at least one three-dimensional (3D) image;
detecting an injury from the 3D image using the machine learning;
classifying the detected injury using the machine learning; and
assessing a criticality of the detected injury based on the classifying using the machine learning.

2. The method of claim 1, further comprising:
quantifying the classified injury, the assessing assesses the criticality based on the quantifying.

3. The method of claim 2, wherein the quantifying includes,
determining a volume of the detected injury using the machine learning.

4. The method of claim 2, wherein the quantifying includes,
estimating a total blood loss using the machine learning.

5. The method of claim 1, further comprising:
selecting one of a plurality of therapeutic options based on the assessed criticality using the machine learning.

6. The method of claim 1, further comprising:
displaying the detected injury in the image; and
displaying the assessed criticality over the image.

7. The method of claim 6, wherein the displaying the assessed criticality includes providing an outline around the detected injury, a weight of the outline representing the assessed criticality.

8. A system comprising:
a memory storing computer-readable instructions; and
a processor configured to execute the computer-readable instructions to,
determine scan parameters of a patient using machine learning,
obtain a three-dimensional (3D) image of the patient, the 3D image being generated from the determined scan parameters,
detect an injury from the 3D image using the machine learning,
classify the detected injury using the machine learning, and
assess a criticality of the detected injury based on the classification of the detected injury using the machine learning.

9. The system of claim 8, wherein the processor is configured to execute the computer-readable instructions to quantify the classified injury, the assessed criticality being based on the quantification.

10. The system of claim 9, wherein the processor is configured to execute the computer-readable instructions to determine a volume of the detected injury using the machine learning.

11. The system of claim 9, wherein the processor is configured to execute the computer-readable instructions to estimate a total blood loss using the machine learning.

12. The system of claim 8, wherein the processor is configured to execute the computer-readable instructions to select one of a plurality of therapeutic options based on the assessed criticality using the machine learning.

13. The system of claim 8, wherein the processor is configured to execute the computer-readable instructions to,
display the detected injury in the image; and
display the assessed criticality over the image.

14. The system of claim 13, wherein the processor is configured to execute the computer-readable instructions to display the assessed criticality by providing an outline around the detected injury, a weight of the outline representing the assessed criticality.
